(19) United States
(12) Patent Application Publication
(10) Pub. No.: US 2004/0132201 A1
(43) Pub. Date: Jul. 8, 2004

(54) Method for dosing diethanolamine in aqueous solution

(76) Inventors: Jean-Francois Buisson, Martigues (FR); Philippe Macon, Maneglise (FR); Frederic Pouly, Lyon (FR)

Correspondence Address: SUGHRUE MION, PLLC, 2100 PENNSYLVANIA AVENUE, N.W., SUITE 800, WASHINGTON, DC, 20037, US

(21) Appl. No.: 10/469,382

(22) PCT Filed: Mar. 1, 2002
(86) PCT No.: PCT/FR02/00753

(30) Foreign Application Priority Data
Mar. 1, 2001 (AF) R01/02819

(51) Int. Cl. G01N 33/00

(52) U.S. Cl. 436/111; 436/171

(57) ABSTRACT

According to the application, a novel derivative compound is formed from a compound that can react by substitution with diethanolamine. Subsequently, the content of the novel compound is measured by means of ultraviolet spectrometry using the absorbance spectrum of the compound which is measured in a wavelength range included between 200 nm and 350 nm. The content of said novel compound is then converted to the original diethanolamine content.

The present invention concerns a new method for the quick measurement of diethanolamine in aqueous solution. More particularly, the invention concerns a method for measuring the quantity of diethanolamine present in water, usable in the domain of analysis, but also in a continuous process for processing industrial water.

It is known that diethanolamine (abbreviated D.E.A.) has numerous applications in industry, particularly for the desulfurization of gases or for the neutralization of sulfurous molecules contained in gasolines, in desulfurization processes used, for example, in petroleum refineries.

It is also known that D.E.A. contributes strongly to the formation of TOC (total organic carbon) in industrial waste waters and that this molecule acts as a poison for the bacterial beds of the biofilters used to treat said waters prior to their release into the natural environment.

It therefore appears to be indispensable to monitor the evolution of these concentrations of D.E.A. and to be able to measure the quantities as accurately as possible, in order to optimize, for example, the operation of the desulfurization processes, or to monitor the quality of industrial waste waters so as to comply with the very strict environmental standards.

One method of measurement used in the technology requires the use of a separative technique such as in high pressure liquid phase chromatography, after chemical transformation of the D.E.A.

This analysis technique, which involves the use of an expensive apparatus, is not available in all testing laboratories because of its complexity, and in addition it requires a level of expertise to use it. Unfortunately it also takes a long time to obtain results because its analysis time is about an hour, limiting its on-line use all the more.

The present invention seeks to remedy these disadvantages by proposing a technique of measuring by ultraviolet spectrometry, which is simple, faster and easier to implement than the laboratory method discussed above, and in addition, can be adapted to use on line.

The D.E.A. contained in an aqueous solution, however, is not directly observable in the state in ultraviolet, because its absorption spectrum, not very sensitive, is situated in the far-ultraviolet (less than 200 nm), which is also an area of absorption of a plurality of chemical compound such as dissolved oxygen and carbonic gas, as well as carboxylic acids and different hydrocarbons that are normally found in industrial waters.

This is the reason, according to the invention, one begins by forming a new compound from a reagent specific to secondary amines, in which the D.E.A., the concentration of which is to be measured, is substituted for one atom of this reagent. This new compound, which is then detectable by UV spectrometry, is then measured in a known way, by a person skilled in the art, in a band of wavelengths between 200 nm and 350 nm. It is from the concentrations calculated in this new compound that the concentration of D.E.A. in the sample can finally be deduced directly.

Consequently, an object of the invention is a method for measuring the quantity of diethanolamine present in an aqueous solution, characterized in that a new derivative compound is formed from a compound suitable for reacting by substitution with the diethanolamine, the concentration thereof then being measured by ultraviolet spectrometry from the absorption spectrum measured in a range of wavelengths between 200 nm and 350 nm, and in that the concentration of this new compound is converted to the original concentration of diethanolamine.

The new compound formed by substitution of one atom of the reagent with the D.E.A. contained in the sample can be chosen, for example, from the group composed of amides, urethanes and tertiary amines.

In a preferred implementation of the invention, this derivative is advantageously the urethane of D.E.A., or “Fmoc-D.E.A.,” obtained by reaction of the D.E.A. with 9-methylfluorenyl chloroformate, the absorption spectrum of which, measured by ultraviolet spectrometry, has two peaks at wavelengths of 293 nm and 301 nm, respectively.

In a first form of implementation of the method of the invention, it is possible to measure the concentration of a derivative compound of the D.E.A., for example Fmoc-D.E.A., of the sample, when the substitution reaction of the reagent, i.e., the 9-methylfluorenyl chloroformate with the D.E.A. contained in the sample, is completed.

This form of implementation, however, has the disadvantage that the substitution reaction, as indicated above, is relatively slow if the concentration of the reagent is not very high, and it then becomes necessary to agitate under controlled circumstances the reaction mixture, which constitutes a serious operational limitation, particularly on line, such as for example for sweetening gasolines in a petroleum refinery.

A second form of implementation of the invention, which is preferred, no longer consists of measuring the concentration of the compound derived from the D.E.A. after completion of the reaction with the reagent introduced into the sample for that purpose, particularly 9-methylfluorenyl chloroformate, but rather to observe the speed of this reaction by measuring the quantity of derivative formed during a predetermined time interval, in order to deduct therefrom the initial quantity of D.E.A. present in the sample.

The Applicant has established that if the ultraviolet absorption spectrum of the reaction medium is recorded at to and to+t, the absorption difference A for one of the characteristic peaks of the recorded spectrum, for example for one and/or the other of the peaks at 293 nm and 301 nm for the Fmoc-D.E.A., is in linear relation with the sample's initial concentration C of D.E.A.

In other words, C=aA+b where a and b are constants, so that by measuring the absorption at times to+t for at least one absorption peak as indicated above of the UV spectrum, it is possible to deduct, directly and simply, the sample's initial concentration of D.E.A. from the difference A.

This preferred form of implementation of the method covered by the invention is therefore carried out in the following way:

the compound intended to react with the D.E.A. contained in the sample is introduced therein in order to produce the derivative directly observable by ultraviolet spectrometry;

the ultraviolet absorption spectra of the sample containing the derivative of the D.E.A. is then recorded at times to and to+t;

the absorption at times to and to+t is measured for at least one of the characteristic peaks of the recorded spectra;

the absorption difference A between the times to+t and to;

and by calculation the sample's initial concentration C of D.E.A. is deduced from this difference A by applying the relation C=aA+b, where a and b are predetermined constants.

All of these operations can be performed within a very short time, on the order of seven to eight minutes, without the need for complex and costly equipment or a person who is an analysis expert, and this represents a considerable advantage of the method according to the invention compared to methods currently in effect.

Preferably, the reaction medium is kept at a controlled pH of between 8 and 9, and more preferably still, appreciably equal to 8.5. The method according to the invention has the advantage of being insensitive to the presence of various impurities in the analyzed sample, particularly phenols or sulfurs, that are normally found in industrial waste waters from a petroleum refinery. Furthermore, these impurities have no affect on the kinetics of the substitution reaction and do not change the absorption of the UV spectrum peaks with which the present method is concerned.

However, the possible presence of parasite compounds such as secondary or primary amines contained in the sample can disturb the measurements, and can cause, for example, the appearance of a parasitic peak in the measured spectra.

In order to remedy this drawback, it is necessary to proceed with a deconvolution of the spectrum or spectra obtain by ultraviolet spectrometry, that is, to break them down into elementary reference spectra. These reference spectra in most cases correspond to the spectra obtained in “pure” compounds for which the concentrations are known. The deconvolution of the sample's spectrum will make it possible to determine the contribution of each of the reference spectra and therefore to mathematically determine the quantitative composition of the sample. The spectra of so-called calibration samples, and consequently of known compositions, can be contained in a computer memory, and the spectral deconvolution operation can thus be conducted with the help of software programs that are known in the technology, thus making it possible to obtain directly the analysis results without operator intervention.

The Examples provided below illustrate the implementation of the invention of course, they are not limiting in nature.

The results obtained in these Examples are illustrated by the attached drawings, in which:

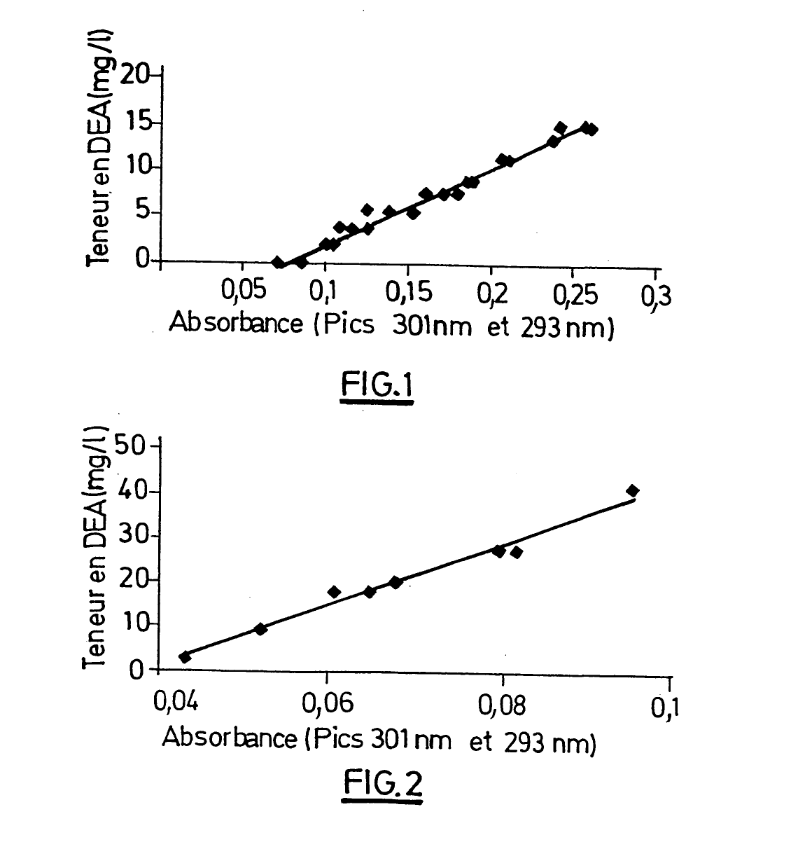

FIG. 1 is a diagram showing the linear correlation between the concentration of D.E.A. in the samples used in the Example 1 and the sum of the absorptions respectively measured at 293 nm and 301 nm on spectra obtained by ultraviolet spectrometry of a derivative of D.E.A., and more specifically of Fmoc-D.E.A., resulting from an addition to these samples, according to the invention, of 9-methylfluorenyl chloroformate;

FIG. 2 is a diagram similar to the one in FIG. 1 for waste water samples produced by a purification treatment at the outlet of a unit for clarifying these waters and used in Example 2, to which known quantities of D.E.A. have been added;

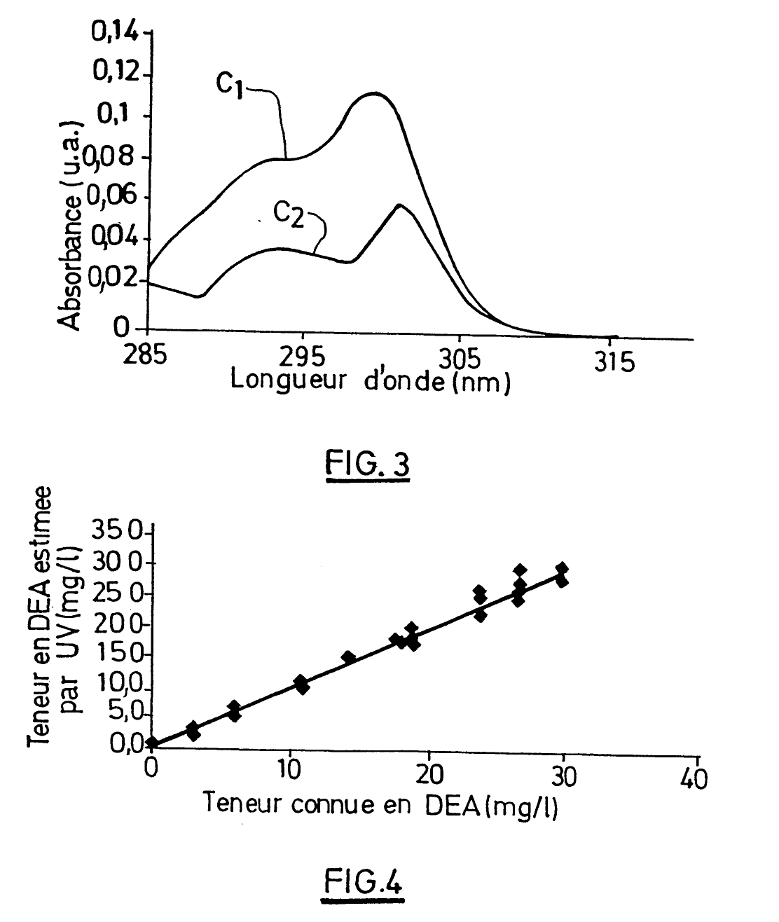

FIG. 3 is a diagram illustrating two reference spectra used in ultraviolet spectrometry, respectively referenced as “C1” and “C2,” and constituting a deconvolution base suitable for the use of the reagent according to the invention;

FIG. 4 is a diagram showing the correlation obtained between the known D.E.A. concentrations and those same concentrations obtained by the measuring method according to the invention, the samples used to draw the correlation curve being from the industrial waters used in the Example 3, taken in a water treatment process of a refinery, free of D.E.A. at the source and into which known quantities of D.E.A. have been added.

### EXAMPLE 1

This Example is intended to illustrate the implementation of the method according to the invention on demineralized water samples that have had know quantities of D.E.A. added.

These samples are buffered to obtain a pH appreciably equal to 8.5 with a solution comprised essentially of di-sodium tetraborate and hydrochloric acid. The pH of the samples is verified before implementing the method according to the invention and should be maintained between 8 and 9 by additions of soda or sulfuric acid.

A solution of 9-methylfluorenyl chloroformate is then added in sufficient quantity to obtain a concentration of this reagent equal to 150 mg/l. This concentration is weak enough so as not to saturate the ultraviolet spectra, which will be measured in the area of wavelengths of 285 nm to 320 nm, but high enough to allow the reaction of this compound with the D.E.A., in order to obtain a derivative detectable by its absorption peaks at 293 nm and 301 nm.

The spectrum of the reaction mixture contained in a cuvette, such as quartz, having an optical path 10 millimeters long, is recorded in this band of wavelengths, in accordance with the recommendations for the technique of ultraviolet spectrometry analysis. This recording takes place twice, first one minute after mixing with the 9-methylfluorenyl chloroformate, then again five minutes after the first recording. In a known way, the spectrum obtained after one minute of reaction is subtracted from the one obtained after six minutes of reaction in order to obtain a new spectrum representative of the quantity of Fmoc-D.E.A. formed in five minutes of reaction. In FIG. 1, the known quantities of D.E.A. of the samples (in mg/l) are shown as ordinates, and the sum of the absorbances measured at 293 nm and 301 nm as abscissas.

It will be noted that there is an appreciably proportional relationship between the initial concentration of D.E.A. in the samples and the development, in the time interval separating the two spectra (5 minutes), of the sum of measured absorbances, reflecting the formation of the derivative of the D.E.A.

The correlation between the initial concentration Y of D.E.A. and the sum of the measured absorbances X can be expressed by the following equation:

y=88.093×−6.7987

with a coefficient of determination R2 appreciably equal to 0.98.

It will be noted that, in order to perform this correlation, the absorbances corresponding to the two peaks of the spectrum, and not just one, have preferably been taken into consideration and summed up in order to make the results obtained reliable. In fact, the D.E.A. concentration could be correlated with just the absorbance measured at 301 nm, but the results obtained would then be more disbursed around the low order of correlation and the accuracy thereof would be lower. This dispersion of experimental readings is primarily due to the fact that the measurements are made in the domains of absorbance close to the limits of sensitivity of the equipment used, in this instance the Senior Anthelie ultraviolet spectrometer marketed by Secomam. The additional information obtained by also the absorbance at 293 nm as well makes it possible to minimize the error due to the equipment itself.

It should be noted, however, that for a zero quantity of D.E.A. an absorbance at 293 and 301 nm is still observed. These absorbances are from the Fmoc-OH formed by hydrolysis during the reaction of the D.E.A. with the 9-methylfluorenyl chloroformate. Because the pH of the sample is maintained constant, however, the hydrolysis produces in reaction the same quantity of Fmoc-OH and the contribution of this product to the ultraviolet spectrum, although small, is constant. In order to obtain the quantity of D.E.A., therefore, the contribution of the Fmoc-OH to the absorbances concerned is simply subtracted.

In order to determine whether the presence of phenols and sulfurs in the samples affects the kinetics of the reaction between the D.E.A. and the 9-methylfluorenyl chloroformate, 10 mg/l of phenols and 10 mg/l of sulfurs (HS) are added alternatively to the treated samples, always with a pH controlled at 8.5. In no case was the absorbance of the measured spectra modified.

### EXAMPLE 2

This Example concerns analyses performed on industrial waste water samples free of D.E.A., issuing from a clarification treatment of said water, and to which known quantities of D.E.A. have been added.

The procedure is identical to the tests in Example 1, by incorporating the D.E.A. into the samples removed and by making the same absorbance measurements by ultraviolet spectrometry.

The results obtained are presented in FIG. 2, where the concentrations of D.E.A., expressed in mg/l, appear as ordinates and the sum of the absorbances for the peaks at 293 nm and 301 nm appear as abscissas.

It will be noted that, as in the Example 1, the points are distributed appreciably along a correlation line, the equation for which is as follows:

y=705.91×−27.418

with a coefficient of determination appreciably equal to 0.99.

These results show that with the method according to the invention it is possible to make problem-free measurements of the D.E.A. in waste water samples issuing from clarification.

For actual concentrations of D.E.A. varying from 0.0 to 15.6 mg/l, the standard deviation between the measured values and the actual values varies from 0.2 to 0.5.

### EXAMPLE 3

This example concerns measuring the D.E.A. in waste waters in the process of purification, which are obtained at the intake of the treatment of waters not containing D.E.A., to which known quantities of D.E.A. are added. The samples are treated identically to those in Example 1, that is, by reaction with 9-methylfluorenyl chloroformate and recording of the spectra obtained by ultraviolet spectrometry of the reaction mixture, one minute and six minutes after producing said mixture. The measured spectra present a large peak at 299 nm, thus revealing a parasitic reaction, probably due to the presence of secondary or primary amines in the waters of the reaction medium.

It is necessary, therefore, to break down by deconvolution the UV spectra obtained between 285 and 320 nm, in order to better separate the contribution of the derivative having the D.E.A., that is, the Fmoc-D.E.A., from those of the other reaction products, i.e., the hydrolysis product of the reaction, the Fmoc-OH, and the parasite product or products as mentioned above.

In the present case, the hydrolysis product and the parasite product or products have been regrouped into a single reference spectrum (curve C1 of FIG. 3, also showing the curve C2 representative of the spectrum of the derivative of the D.E.A.).

The coefficient of contribution of this derivative after deconvolution can then be correlated with the initial concentration of D.E.A. in the samples, plus known quantities of D.E.A.

The results obtained are shown in FIG. 4, where the estimated concentration of D.E.A. in mg/l using a method according to the invention is indicated as ordinates and the actual concentration x, also in mg/l, appears as abscissas.

Here again it will be noted that there is a linear relationship between x and y, the correlation line being in accordance with the equation:

y=0.9929×+0.0781

with a coefficient of determination appreciably equal to 0.99.

The tests reported in the Examples above show the great reliability of the method according to the invention and its ease of implementation. Moreover, the application of known means in the technology does not pose any difficulty of the on-line use of the method according to the invention.

1. Method for measuring the quantity of diethanolamine present in an aqueous solution, characterized in that a new derivative compound is formed from a compound suitable for reacting by substitution with the diethanolamine, the concentration thereof then being measured by ultraviolet spectrometry from the absorption spectrum measured in a range of wavelengths between 200 nm and 350 nm, and in that the concentration of this new compound is converted to the original concentration of diethanolamine.

2. Method according to claim 1, characterized in that the new derivative compound is formed by substitution of one atom from the reagent with the diethanolamine contained in the solution.

3. Method according to claim 2, characterized in that the reagent is chosen from the group composed of amides, urethanes and tertiary amines.

4. Method according to claim 2, characterized in that the new derivative compound formed by substitution of one atom from the reagent with the diethanolamine contained in the sample is the urethane of diethanolamine or Fmoc-D.E.A.

5. Method according to claim 4, characterized in that the compound suitable for reacting with the diethanolamine to form the urethane of diethanolamine is 9-methylfluorenyl chloroformate.

6. Method according to any of the preceding claims, characterized in that the absorption of the Fmoc-D.E.A. is measured at two characteristic peaks at wavelengths of 293 nm and 301 nm.

7. Method according to claim 6, characterized in that the absorptions measured at 293 nm and 301 nm are added together.

8. Method according to any of the preceding claims, characterized in that the absorption is measured at wavelengths of 293 nm and 301 nm after complete reaction of the reagent and the diethanolamine.

9. Method according to any of the preceding claims, characterized in that:
the compound intended to react with the diethanolamine is introduced into the sample containing said diethanolamine in order to produce the derivative directly observable by UV spectrometry;
the UV spectra of the sample containing the derivative of the diethanolamine are then recorded for times to and to+t;
the absorption is measured at times to and to+t for at least one of the characteristic peaks of recorded spectra;
the difference A of absorption between the times to+t and to is determined;
the initial concentration C of diethanolamine in the sample is calculated from this difference A, by applying the formula C=aA+b, where a and b are predetermined constants.

10. Method according to any of the preceding claims, characterized in that the reaction medium is maintained at a pH controlled between 8 and 9.

11. Method according to claim 10, characterized in that the reaction medium is maintained at a pH controlled appreciably equal to 8.5.

12. Method according to any of the preceding claims applied to a sample containing parasite compounds such as secondary or primary amines, that could affect the spectrum or spectra obtained by ultraviolet spectrometry, characterized in that said spectra are deconvoluted, that is, they are broken down into elementary spectra of a certain number of pure compounds present and the spectra obtained are compared to those of samples of known composition containing the same compounds;

13. Method according to any of the preceding claims, applied to a process of continuous treatment of water from a petroleum refinery.

14. Method according to any of claims 1 to 11, applied to the treatment of water issuing from the sweetening of petroleum cuts in a petroleum refinery.
